(19) United States
(12) Patent Application Publication
(10) Pub. No.: US 2023/0320671 A1
(43) Pub. Date: Oct. 12, 2023

(54) SPIROXMETER SMART SYSTEM

(71) Applicant: HD Data Incorporated, Las Vegas, NV (US)

(72) Inventors: Dwight Anson YORKE, Las Vegas, NV (US); Henry ROSKOS, Las Vegas, NV (US)

(21) Appl. No.: 18/205,785

(22) Filed: Jun. 5, 2023

Related U.S. Application Data
(62) Division of application No. 15/816,902, filed on Nov. 17, 2017, now abandoned.

(51) Int. Cl. A61B 5/00; A61B 5/1455; G16H 40/63; G16H 10/60; A61B 5/087

(52) U.S. Cl. CPC A61B 5/7275; A61B 5/0022; A61B 5/087; A61B 5/14551; G16H 10/60; G16H 40/63; A61B 2560/0242; G16H 50/20

(57) ABSTRACT

Systems, devices, and methods for administering healthcare to a user or patient based on analysis of user-specific respiratory and oximetry parameters is disclosed. Additional parameters specific to the user, the user’s health condition, or the user’s current or planned treatment are optionally included in the analysis. The user inputs parameter values on a healthcare monitoring device. The device communicates such values to a healthcare information hub, which pre-processes the values into data sets and forwards the data sets to a healthcare server. The hub performs preliminary analysis of the parameter values and provides a preliminary report to the user that indicates a characterization of the user’s health condition (e.g., diagnosis, prognosis, suggested preliminary therapy regimen, etc) or whether further parameter values should be input. The healthcare server analyzes the datasets received from the information hub, optionally including additional data sets (e.g., environmental parameters local to the user, demographic data related to the user, the user’s medical history, etc), and infers an instruction for the user. The instruction is relayed to the patient via the information hub and directs the patient to use the monitoring device to obtain further respiratory or oxygenation parameter values, follow a therapy regimen for the user’s health condition, or various partial or complete combinations of both.

This application is a divisional of application ser. no. 15/816902, filed Nov. 17, 2017, the disclosure of which is incorporated herein by reference.

### FIELD OF THE INVENTION

The field of the invention is methods, systems, and devices for personalized health regimens and monitoring.

### BACKGROUND

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Personal devices used to monitor biometric data are quite popular and offer a variety of services. Some, such as the Fitbit® or smartphone accessories, monitor activity levels and cardiovascular metrics, and are well suited for the amateur athlete or curious consumer. While such devices can provide interesting data to fitness enthusiasts, they are not well suited for monitoring a user’s health conditions, detecting ailments, or suggesting regimens to improve the user’s health or relieve an ailment.

However, there are devices known and available to specifically monitor a user’s health condition based on various biometric variables. For example, blood glucose meters are used by patients with diabetes and blood pressure monitors are used by patients with heightened risk for heart disease. However, many of these devices do not have Internet of Things (“IoT”) connectivity, and therefore lack the improved efficiencies, new capabilities, and scalability of services attainable with IoT devices.

Efforts have been made to bring the IoT to personal devices designed to monitor a user’s health condition via biometric variables. For example, Medical International Research’s Spirodoc® device (see US 2013/0184540) allows users to monitor their respiratory function, blood oxygen saturation, and physical activity. The data collected by the device can then be uploaded to a web server to allow a doctor to review the data and provide therapy instructions to the user. However, such devices fail to leverage the computing power of smartphones, fail to provide instantaneous feedback to the user, fail to consider extraneous, user specific variables or metrics, and are dependent upon analysis by medical professionals. Each of these failures present an especially compounded problem for users with acute conditions that are triggered by user specific circumstances that require instantaneous analysis regardless of place or time.

Thus, there remains a need for a system and method that measures multiple biometric variables of a user and provides instantaneous feedback that is specific to the user at that specific place and specific time.

### SUMMARY OF THE INVENTION

The inventive subject matter provides apparatus, systems, and methods for administering healthcare to a user or patient based on user specific variables. The variables include a respiratory parameter and an oxygenation parameter, and can include further parameters specific to the patient or the patient’s surroundings. The variables are used to generate a preliminary report and instructions for the patient to take further measurements or to follow a therapy regimen.

In one embodiment, a system for administering healthcare to a patient includes a patient monitoring device. The monitoring device has both a spirometer programmed to determine a value for the patient’s respiratory parameter and a pulse oximeter programmed to determine a value of the patient’s oxygenation parameter. In some embodiments, the patient monitoring device determines at least three parameters of a pulmonary function test (PFT), and includes additional components for determining such parameters as necessary. It is contemplated that the monitoring device is designed to be operated by the patient. In some embodiments, the patient monitoring device further includes at least one of an acoustic sensor programmed to determine a value for an acoustic parameter, a barometric pressure sensor programmed to determine a value for a barometric pressure parameter, or a humidity sensor programmed to determine a value for a humidity parameter.

The system further includes a healthcare information hub. The healthcare information hub is communicatively coupled with the monitoring device and is programmed to receive data from the monitoring device. Once received, the healthcare information hub uses the data to generate a preliminary report (and optionally exploratory data analysis) and to deliver the report directly to the patient. It is contemplated the preliminary report typically includes a characterization (e.g., a diagnosis, etc) or a forecast (e.g., a prognosis, etc) of the patient’s disease condition.

The system further comprises a healthcare server. In some embodiments, the healthcare server is programmed to store at least one of the value for the respiratory parameter, the value of the oxygenation parameter, a further respiratory parameter value, a further oxygenation parameter value, or the therapy regimen. In preferred embodiments, the healthcare server is programmed to compile at least some of the patient specific data it analyzes into an electronic medical record (EMR) for the specific patient.

The healthcare server is programmed to infer and deliver an instruction to the patient based on at least one of the respiratory parameter value, the oxygenation parameter value, and the preliminary report. In preferred embodiments, the healthcare server infers an instruction further based on at least one of an environmental data local to the patient, a historical health data of the patient, and a health data of the patient’s demographic.

It is contemplated the instruction directs the patient to at least use the device to obtain a further respiratory or oxygenation parameter value, or to follow a therapy regimen for the disease condition. In some embodiments the patient monitoring device (or the healthcare information hub) is configured to alert the patient to an error in one of the spirometer, the respiratory parameter value, the pulse oximeter, or the oxygenation parameter value. The instruction can also direct the patient to self-administer the therapy regimen, refer the patient to a healthcare professional, or both.

Further, it is contemplated all communication between the patient monitoring device, the healthcare information hub, and the healthcare server is compliant with the Health Insurance Portability and Accountability Act (HIPAA), as some information gathered and analyzed via the system is highly sensitive personal health data.

The inventive subject matter further contemplates methods of administering healthcare to a patient. In one step, a patient monitoring device measures a respiratory parameter and an oxygenation parameter of the patient. Generally, the respiratory parameter value is derived from a spirometer, and the oxygenation parameter value is derived from a pulse oximeter. The values of the measured parameters are received by a healthcare server and used to generate and deliver an instruction to the patient to perform a respiratory therapy. It should be appreciated that the instruction can include additional information, such as a preliminary analysis of the parameters.

After the patient performs the respiratory therapy, the patient monitoring device measures an additional parameter selected from either a respiratory parameter or an oxygenation parameter (or both) of the patient. The additional parameter value(s) is received by the healthcare server and used to generate a report including at least one of (a) a characterization, a forecast, or a therapy of a disease, or (b) an instruction to perform a test. Further, the healthcare server preferably filters out false positive results (e.g., a characterization, a forecast, or a therapy of a disease) based on at least one of an environmental data local to the patient, a historical health data of the patient, or a health data of the patient’s demographic. In preferred embodiments, the healthcare server delivers the report to the patient.

The inventive subject matter further contemplates a patient monitoring device including a spirometer and a pulse oximeter, each of which is informationally coupled to a processor. In some embodiments, the device also includes at least one of an acoustic sensor, a barometric pressure sensor, or a humidity sensor informationally coupled to the processor. The device also has a validity indicator informationally coupled to the processor. The validity indicator alerts a user to an error with data from the spirometer or the pulse oximeter. In preferred embodiments, the validity indicator triggers an instruction to the patient to cure the error.

The patient monitoring device further includes a user interface informationally coupled to the processor. The user interface displays a summary of readings from the spirometer and the pulse oximeter, and in preferred embodiments displays the instructions to the patient.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 shows an example of a system architecture for administering healthcare to a patient.

FIG. 2 shows a flow chart for an example method for administering healthcare to a patient.

FIG. 3 shows a block diagram or an example device for monitoring the health of a patient.

FIGS. 4A-4C shows several conformations of an example device for monitoring the health of a patient.

FIGS. 5A-5C shows several conformations of another example device for monitoring the health of a patient.

FIGS. 6A-6C shows several conformations of a further example device for monitoring the health of a patient.

### DETAILED DESCRIPTION

The inventive subject matter provides apparatus, systems, and methods for administering healthcare to a user or patient based on user specific variables. The variables include at least a respiratory parameter and an oxygenation parameter, and can include additional parameters specific to the patient (e.g., environmental parameter, personal health parameter, demographic parameter, pharmaceutical parameter, etc), the patient’s condition (e.g., symptoms, prognosis, mortality rate, potential treatments, etc), or the patient’s current or planned treatment (e.g., pharmaceutical regimen, inhaler, physical therapy regimen, diet, side effects, success rate, health diagnostics, monitoring devices, etc). The variables are used to generate a preliminary report (e.g., via a healthcare hub) and instructions (e.g., via a healthcare server) for the patient to take further measurements or to follow a therapy regimen.

The following description includes information that may be useful in understanding the present invention. It is not an admission that any of the information provided herein is prior art, necessary, or relevant to the presently claimed invention, or that any publication specifically or implicitly referenced is prior art.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be noted that any language directed to a computer device or a computer system should be read to include any suitable combination of computing devices, including servers, interfaces, systems, databases, agents, peers, engines, controllers, or other types of computing devices operating individually or collectively in a networked environment (e.g. local intranet or an Internet cloud). One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

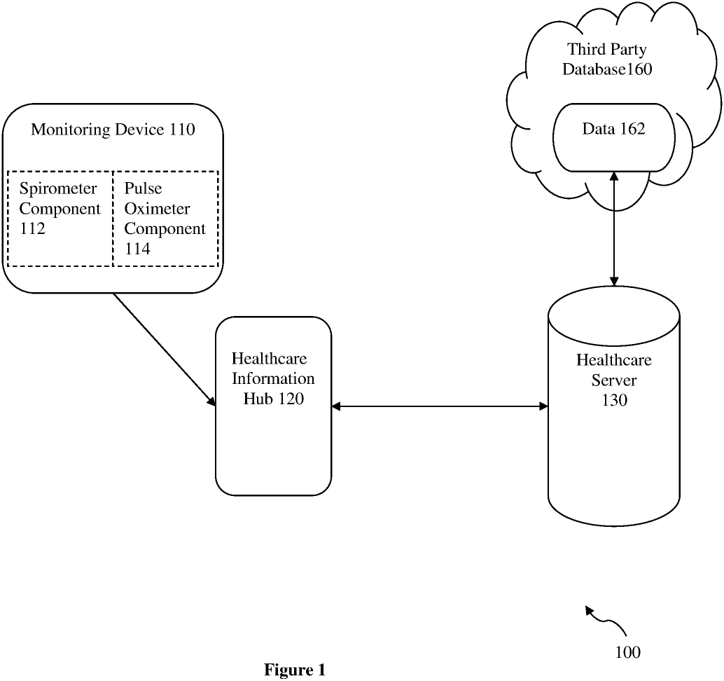

The inventive subject matter provides apparatus, systems, and methods for administering healthcare to a user or patient based on user specific variables. In one embodiment (e.g., FIG. 1), a system 100 administers healthcare to a patient and includes monitoring device 110, healthcare information hub 120, and healthcare server 130.

Monitoring device 110 monitors a patient, and has both spirometer component 112, which is programmed to determine a value for the patient’s respiratory parameter, and pulse oximeter component 114, which is programmed to determine a value of the patient’s oxygenation parameter. In some embodiments, the patient monitoring device determines at least three (or more than 5, 7, 10, or 15) parameters of a pulmonary function test (PFT) (e.g., forced expiratory volume in one second (FEV1), forced vital capacity (FVC), ratio of FEV1/FVC, forced expiratory flow (FEF), peak expiratory flow rate (PEF) forced inspiratory flow rates (FIFs), maximal voluntary ventilation (MVV), tidal lung volume (VT), inspiratory reserve lung volume (IRV), expiratory reserve lung volume (ERV), residual lung volume (RV), total lung capacity (TLC), inspiratory capacity (IC), functional residual capacity (FRC), vital capacity (VC), maximal inspiratory pressure (MIP), maximal expiratory pressure (MEP), single-breath diffusing capacity for carbon monoxide (DLCO), oxygen saturation (SO₂) monitoring, oxygen desaturation during exercise (six-minute walk test), arterial blood gases (ABGs), peripheral arterial oxygen saturation (SpO₂), etc), and includes additional components for determining such parameters as necessary.

It is contemplated that monitoring device 110 is highly portable (e.g., less than 10 lbs, 8 lbs, 5 lbs, 4 lbs, 3 lbs, 2 lbs, or 1 lb), or at least mobile (e.g., battery powered, wireless, etc), and is designed to be operated by the patient or a layperson (e.g., if the patient is incapacitated, otherwise unable to operate device, etc). In some embodiments, monitoring device 110 further includes at least one (or two, or three) of (a) an acoustic sensor programmed to determine a value for an acoustic parameter, (b) a pressure sensor (e.g., barometric, etc) programmed to determine a value for a pressure parameter (e.g., barometric pressure parameter, etc), or (c) a humidity sensor programmed to determine a value for a humidity parameter, and may further include a GPS device, an accelerometer, an air quality sensor, a thermometer, a fingerprint reader, or a barometer.

System 100 further includes healthcare information hub 120. Healthcare information hub 120 is communicatively coupled with monitoring device 110, for example wirelessly (e.g., Bluetooth, Wi-Fi Direct, cellular network, NFC, Wi-Fi network, etc) or wired (e.g., USB cord). In some embodiments, monitoring device 110 is a peripheral hardware device (e.g., spirometer with pulse oximeter) that attaches to healthcare information hub 120 (e.g., healthcare information hub 120 is a smartphone, and monitoring device 110 attaches via a USB port). Healthcare information hub 120 is programmed to receive (via communicative coupling) data from monitoring device 110. Such data generally includes respiratory or oximetry parameter values, and can also include additional data relevant to the patient’s health (e.g., data input by the patient such as symptoms, conditions, medical history, demographic data, user preferences, user budget, user location, environmental conditions local to user, etc)

Once received, healthcare information hub 120 uses the data to generate a preliminary report (e.g., health assessment, condition assessment, warning, emergency instructions, initiate emergency contact, instruction for repeat measurements, etc) and to deliver the report directly to the patient (e.g., via a display on information hub 120, a display on monitoring device 110, etc). It is contemplated the preliminary report typically includes a characterization (e.g., a diagnosis, a severity, a stress level of the disease condition, etc) or a forecast (e.g., a prognosis, etc) of the patient’s disease condition. Viewed from another perspective, the preliminary report can include an intuitive or simplistic summary of the data, such as that the patient’s pulse, blood oximeter, and spirometer results show the patient is in a healthy condition, or indicate a stressed condition, experiencing an episode of a respiratory condition (e.g., asthma attack, etc), or that the patient is likely to experience an episode. In emergency situations, information hub 120 may contact emergency services (e.g., 9-1-1) or a hospital.

Information hub 120 can optionally perform exploratory data analysis of the received or compiled data before forwarding the data to healthcare server 130 (e.g., box plot, histogram, multi-variable chart, run chart, Pareto chart, scatter plot, stem-and-leaf plot, parallel coordinates, odds ratio, multidimensional scaling, targeted projection pursuit, principal component analysis (PCA), multilinear PCA, dimensionality reduction, nonlinear dimensionality reduction (NLDR), projection methods such as grand tour, guided tour and manual tour, median polish, Trimean, ordination, etc).

It should be appreciated that healthcare information hubs of the inventive subject matter can be any device with requisite memory, processor, and wireless communication hardware, but preferably the hub is the patient’s smartphone device (e.g., an application on the smartphone, an accessory attachment to the smartphone, a web application accessed on the smartphone, etc). It should be apparent that using the patient’s smartphone device as an information hub allows greater front-end processing and analysis of data from the monitoring device before the data is transmitted downstream, as well as multimedia display capabilities for presenting reports and instructions to the user. Further, as the user’s smartphone likely has ample memory, processing power, and wireless communication abilities, using a smartphone as a hub allows the monitoring device to be made out of lower performance, and lower cost, components. Similarly, as smartphones typically include additional hardware such as accelerometers, GPS devices, large batteries, and USB ports for peripheral devices, using smartphones as the hub allows the monitoring device to leverage such additional hardware resources. Further, as smartphones are generally ubiquitously connected to the internet, using a smartphone allows a simple way to push software updates to the information hub (e.g., application) and the monitoring device (e.g., via communication session with hub). Likewise, as smartphones are excellent communication devices, using smartphones as the information hub enables the system to quickly, and in some cases automatically, contact emergency response services if the user requires immediate medical attention, and inform such services of the patient’s location via GPS or other location sensors on the smartphone.

System 100 further comprises healthcare server 130. It should be appreciated that a single proprietary server, a network of servers, or third party server space (e.g., AWS, Azure, etc) can be used as healthcare server 130. Healthcare server 130 is communicatively coupled to information hub 120. It should be appreciated that healthcare server 130 can also be communicatively coupled directly to monitoring device 110. In preferred embodiments, any communication sessions between healthcare server 130 and healthcare information hub 120 (or between healthcare information hub 120 and monitoring device 110) are encrypted and secure (e.g., via VPN, security key, HIPAA compliant, etc). In some embodiments, healthcare server 130 is programmed to store at least one (or two, or three, or four, or five) of the value for the respiratory parameter, the value of the oxygenation parameter, a further respiratory parameter value, a further oxygenation parameter value, or a therapy regimen, and can also store some or all of any additional patient related data and location dependent environmental data for the patient’s real time, specific location. In preferred embodiments, healthcare server 130 is programmed to compile at least some (preferably all) of patient specific data and analysis into an electronic medical record (EMR) for the specific patient.

Healthcare server 130 is programmed to infer and deliver an instruction to the patient based on at least one (or two, or three, or four) of a respiratory parameter value, an oxygenation parameter value, an environmental value (e.g., specific to patient’s real time location, etc) and the preliminary report from healthcare information hub 120. It should be appreciated that the inference of instructions by the healthcare servers of the inventive subject matter can include automated analysis (e.g., classical statistics, error statistics, Bayesian statistics, likelihood-based statistics, Akaikean-Information Criterion (AIC) based statistics, machine learning algorithms, trained or untrained algorithms/models, algorithms/models trained one or more of data specific to a patient, to a condition, to a disease, to a therapy, to a demographic, to an environmental factor, etc, failsafe parameter thresholds, etc), manual analysis (e.g., analysis of parameters by medical professional, etc), or both. Such analysis is performed on a variety of data sets, including data sets input by the user (e.g., respiratory parameter, oxygenation parameter, patient heart rate, partial pressure of oxygen (PaO₂) in blood, partial pressure of carbon dioxide (PaCO₂) in blood, blood pH, bicarbonate in blood, oxygen content (O₂CT) in blood, oxygen saturation (O₂Sat) in blood, etc) or historical data sets (e.g., patient specific data sets collected over time by monitor device, patient-specific data sets received from healthcare database, data sets representative of a demographic associated with the specific patient, data sets representative of the specific patient’s condition, data sets representative of treatment outcomes associated with the specific patient, etc). It is contemplated such analysis optionally includes data sets from third parties (e.g., atmospheric/environmental conditions from live database, pharmacological data for patient’s medication or potential medication, emergency response assets or healthcare facilities in patient’s local vicinity, etc).

The analysis of data sets can include comparing each set (or a grouping of data sets) to other data sets (e.g., a data set collected from the patient, a data set that identifies the patient (e.g., breathing pattern, breathing noise, other respiratory signature, etc), a standard-healthy data set, a standard-diseased data set, a standard-therapeutic data set, a standard-emergency data set, etc), comparing each value (or a group of values) in a data set (or a grouping of data sets) to other values (e.g., threshold values for a healthy characterization (prognosis, diagnosis, etc), a diseased characterization, or an emergency characterization; standard-gradation of values indicating severity of a condition), or trending of values in the data sets to make predictive characterizations (e.g., prognosis, success of current therapy, success of potential therapy, etc). In preferred embodiments, healthcare servers of the inventive subject matter infer an instruction further based on at least one (or two, or three, or four) of an environmental data local to the patient (e.g., real time, contemporary, historical, combinations, etc), a historical health data of the patient, or a health data of the patient’s demographic. It should be appreciated that systems, methods, and devices of the inventive subject matter are particularly effective at diagnosis and prognosis of respiratory related conditions (e.g., asthma, allergies, chronic bronchitis, respiratory infections, lung fibrosis, bronchiectasis, chronic obstructive pulmonary disease (COPD), emphysema, asbestosis, sarcoidosis, scleroderma, pulmonary tumor, lung cancer, neuromuscular disorders, etc).

It is contemplated the instruction sent by healthcare server 130 to healthcare information hub 120 directs the user (e.g., patient) to use monitoring device 110 to obtain a further respiratory or oxygenation parameter value, follow a therapy regimen for the user’s disease condition, or various partial or complete combinations of both. For example, the instructions can direct a patient to obtain further parameter values when the analysis by healthcare server 130 (or healthcare information hub 120) (a) yields inconclusive results (e.g., caused by user error inputting parameter value, device error, system error, lack of data to form opinion, inconsistent parameter values, etc.), (b) indicates a need for follow-up values (e.g., determine values after following therapy, after prescribed time lapse, more values needed to infer trend, more values needed to homogenize data, etc), (c) indicates a health condition that requires further monitoring (e.g., values identify at-risk patient (or critical, emergency), negative trending condition, possible new condition, unidentified condition, negative (trending/expected) environmental conditions, etc), or (d) otherwise indicates a need for additional data/values. Thus, in some embodiments, patient monitoring device 110 (or healthcare information hub 120) is configured to alert the user (e.g., patient) to an error in one (or more) of spirometer component 112, the respiratory parameter value, pulse oximeter component 114, or the oxygenation parameter value.

Likewise, the instructions sent by healthcare server 130 can direct the user (patient) to follow a therapy regimen for the disease condition when the analysis by healthcare server 130 shows (a) a satisfactory therapy for the condition (e.g., confidence interval of therapy improving the patient’s condition is greater than 50%, 60%, 75%, 85%, 95%, or 98%), (b) a disease condition level that requires immediate attention (e.g., condition related attack pending, patient undergoing condition attack, patient’s life is at risk), (c) the condition level requires pre-therapy conditioning (e.g., fasting, liquid diet, administration of drugs, etc required before treatment), or (d) otherwise indicates the patient should follow a therapy regimen for the disease condition. In some embodiments, the instruction directs the patient to self-administer the therapy regimen (e.g., drugs, physical therapy, deep breathing exercises, etc), or refers the patient to a healthcare professional, or various partial or complete combinations of both.

It is expected that information gathered and analyzed by systems of the inventive subject matter includes highly sensitive personal health data. As such, it is contemplated all communications between patient monitoring devices, healthcare information hubs, and healthcare servers of the inventive subject matter are compliant with the Health Insurance Portability and Accountability Act (HIPAA).

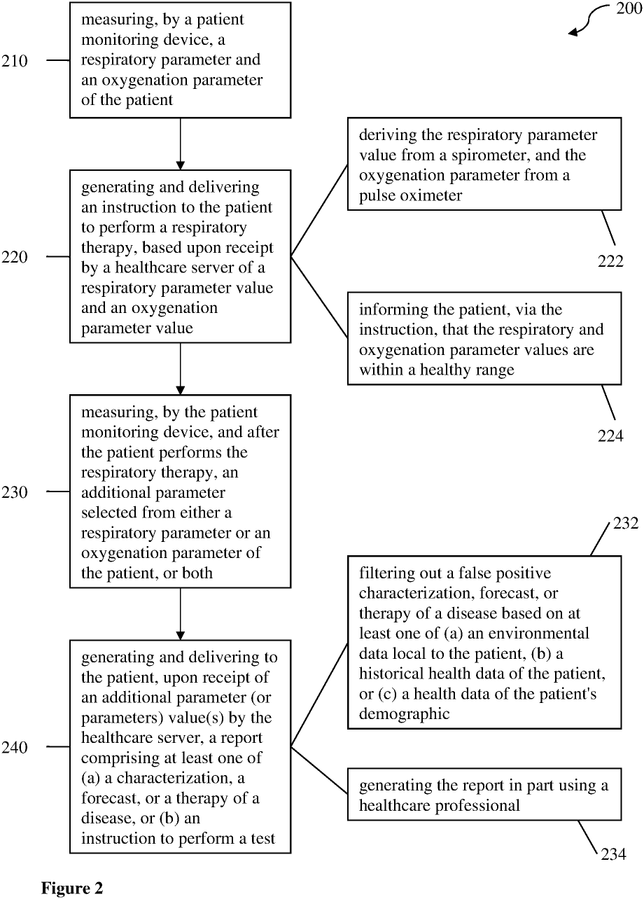

The inventive subject matter further contemplates methods of administering healthcare to a patient (e.g., FIG. 2). In step 210, a patient monitoring device measures a respiratory parameter and an oxygenation parameter of the patient. In some embodiments the respiratory parameter value is derived from a spirometer, and the oxygenation parameter value is derived from a pulse oximeter, as in step 222. In step 220, the values of the measured parameters are received by a healthcare server and used to generate and deliver an instruction to the patient to perform a respiratory therapy. It should be appreciated that the instruction can include additional information, for example as in step 224 informing the patient that the respiratory and oxygenation parameter values are within a healthy range, unhealthy range, normal range for the specific patient, normal range for the patient’s demographic, normal range for the patient’s condition, normal range considering environmental factors, etc, or that the patient should seek emergency health services.

In step 230, after the patient performs the respiratory therapy, the patient monitoring device measures an additional parameter selected from either a respiratory parameter or an oxygenation parameter (or both) of the patient. In step 240, the additional parameter value(s) is received by the healthcare server and used (at least in part) to generate a report including at least one (or all) of (a) a characterization, a forecast, or a therapy of a disease, or (b) an instruction to perform a test. It should be appreciated that the report is generated in part (or whole) by automated analysis (e.g., machine learning algorithm) or by manual analysis (e.g., by a healthcare professional as in step 234). In some embodiments, the healthcare server preferably filters out false positive results (e.g., a characterization, a forecast, or a therapy of a disease) based on at least one (or two, or three) of (a) an environmental data local to the patient, (b) a historical health data of the patient, or (c) a health data of the patient’s demographic, as in step 232. In preferred embodiments, the healthcare server delivers the report to the patient.

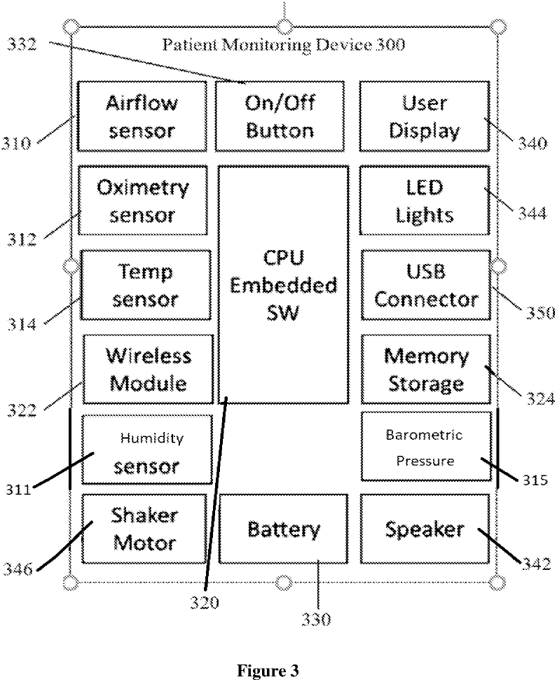

The inventive subject matter further contemplates patient monitoring device 300 including airflow sensor 310 (spirometer), oximetry sensor 312 (pulse oximeter), and temperature sensor 314, each of which is informationally coupled to computer processing unit (CPU) 320 with embedded software, as depicted in FIG. 3. In some embodiments, patient monitoring device 300 also includes at least one (or two, or three, or four) additional sensors, such as an acoustic sensor, a barometric pressure sensor (e.g., 315), or a humidity sensor (e.g., 311) informationally coupled to CPU 320. In preferred embodiments, patient monitoring devices of the inventive subject matter include auditory sensors to detect respiratory sounds of the each specific user (e.g., breathing pattern) in order to identify or confirm the identity of the user. Thus, it should be appreciated that integrating an auditory or acoustic sensor into the spirometer device allows healthcare hubs or healthcare servers to confirm the identity of the patient while the patient is submitting sample data or using the device, as well as providing additional diagnostic information, for example the sound wheezing or raspy breath, which would otherwise go undetected by devices lacking auditory or acoustic sensors. Optionally, patient monitoring device 300 further includes other sensors for monitoring the health or confirming the identity of the patient, such as other spirometry sensors, fingerprint reader, retinal scanner, optical sensor, electrodes (e.g., equivalent circuit model), etc.

Patient monitoring device 300 further includes wireless module 322 for communication with a healthcare information hub or a healthcare server, as well as memory storage 324 for recording and storing sensor data (e.g., from airflow sensor 310, oximetry sensor 312, and temperature sensor 314, etc) or other data input by a user. Patient monitoring device 300 also includes practical components such as battery 330 to power the device and on/off button 332. Patient monitoring device 300 includes communication components, such as user display 340 (e.g., image communication), speaker 342 (e.g., auditory communication), LED lights 344 (e.g., visual communication), and shaker motor 346 (e.g., tactile/vibration communication). USB connector 350 further enables patient monitoring device 300 to receive electrical power from an external charging device (e.g., external battery, main power line, etc), wired communication with peripheral devices (e.g., laptop, external memory, smartphone, etc), or to add peripheral hardware components for further capabilities or upgrades (e.g., GPS device, etc).

In some embodiments, patient monitoring device 300 also has a validity indicator informationally coupled to CPU 320. The validity indicator alerts a user to an error with data from the spirometer or the pulse oximeter. It should be appreciated that such errors could be an excessive reading (e.g., reading beyond a maximum tolerance, beyond a saturation threshold, etc), a faint reading (e.g., reading that does not exceed a minimum tolerance, does not exceed 1:1 signal:noise ratio, etc), a partial reading (e.g., only portion of required interval is measured, partial oximetry reading, partial spirometry reading, etc), a non-patient reading (e.g., non-patient user does not pass biometric security for specific patient, etc), a non-compliant reading (e.g., not taken at instructed time, at instructed setting, in relation to instructed activity, etc), a software error (e.g., glitch, bug, lost memory, etc), or a hardware error (e.g., damaged spirometer, damaged oximeter, insufficient power, obstructed airway, etc). In preferred embodiments, the validity indicator triggers an instruction to the patient to cure the error (e.g., retake oximeter reading, retake spirometer reading, comply with instructions for reading, charge device, repair device, etc) via one of the communication components (e.g., user display 340, speaker 342, LED lights 344, or shaker motor 346). In some embodiments, shaker motor 346 is configured to cause the device to vibrate when a reading is complete, or vibrate to alert the user of an error.

Patient monitoring devices of the inventive subject matter can include less communication components than patient monitoring device 300 (e.g., only user display 340). However, devices of the inventive subject matter can also include additional user interfaces (e.g., visual, auditory, electrical, chemical, biological, tactile, neural, etc) informationally coupled to CPU 320. It should be appreciated that user interfaces (e.g., user display 340) present a summary of readings from the spirometer and the pulse oximeter (or other sensors), and in preferred embodiments provide instructions (e.g., cure reading defect, follow therapy regimen, contact medical professional, etc) to the patient.

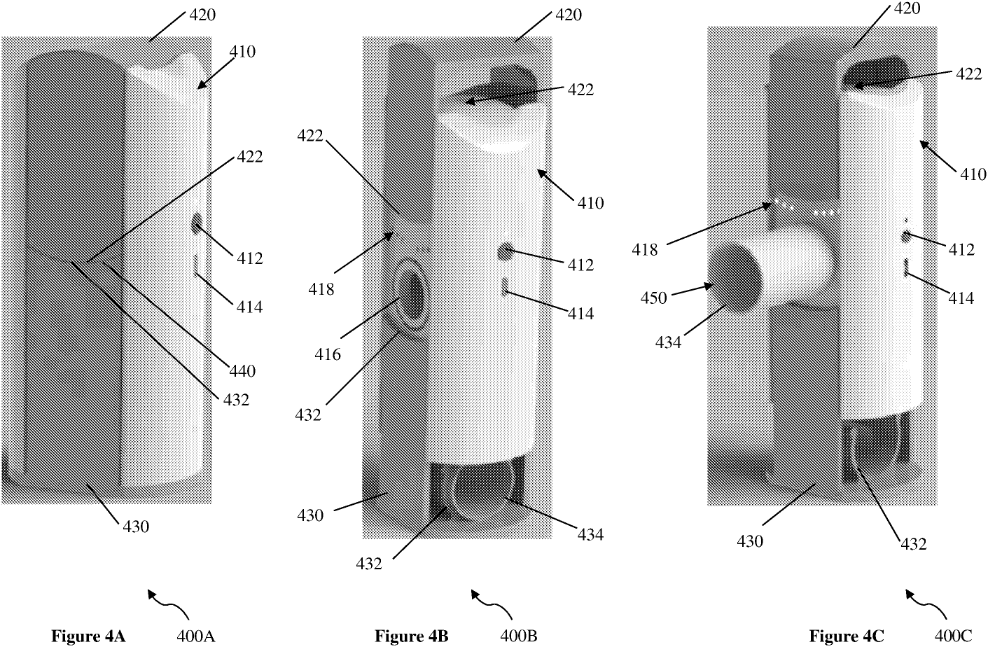

FIGS. 4A-4C depict patient monitoring device 400 in conformation A, B, and C (400A, 400B, and 400C, respectively). Conformation 400A of FIG. 4A depicts a closed conformation of patient monitoring device 400. Device body 410 includes power button 412 (for turning patient monitoring device 400 on and off) and USB port 414 (for charging, wired communication, or adding peripheral attachments to patient monitoring device 400). In conformation 400A, device body 410 can be seen slidingly coupled with housing 420 and housing 430, such that edge 422 of housing 420 and edge 432 of housing 430 meet at interface 440. It is contemplated that conformation 400A is the conformation patient monitoring device 400 will be in when not used by a patient, or when charging via USB port 432. It should be appreciated that in conformation 400A, neither a pulse oximeter nor a spirometer are accessible in patient monitoring device 400.

Conformation 400B of FIG. 4B depicts an open, partially operational conformation of patient monitoring device 400. All similarly numbered components are as described for FIG. 4A. In conformation 400B, housing 420 and housing 430 have each slid away from each other, and from body 410, such that edges 422 and 432 no longer meet at an interface. In conformation 400B, air channel 416 and LED array 418 of body 410 are now exposed, as are pulse oximeter sensor 422, cradle 432, and tube 434. Pulse oximeter sensor 422 is now accessible for a patient to place a finger on sensor to record an oxygenation parameter value. Similarly, tube 434 can now be removed from cradle 432 and inserted into air channel 416 to provide an operational spirometer (e.g., airflow sensor). LED 418 is used to communicate a status of patient monitoring device 400 (e.g., ready for use, sensor in use, data analysis pending, error in data, etc). Thus it should be apparent that patient monitoring device 400 is partially operational in conformation 400B because the pulse oximeter sensor 422 can be used but the spirometer cannot.

Conformation 400C of FIG. 4C depicts an open, fully operational conformation of patient monitoring device 400. All similarly numbered components are as described for FIG. 4A. In conformation 400C, tube 434 is removed from cradle 432 and inserted into air channel 416 to provide an operational spirometer 450. LED array 418 is also fully lit, indicating patient monitoring device 400 is turned on and properly configured for pulse oximeter measurements or spirometer measurements.

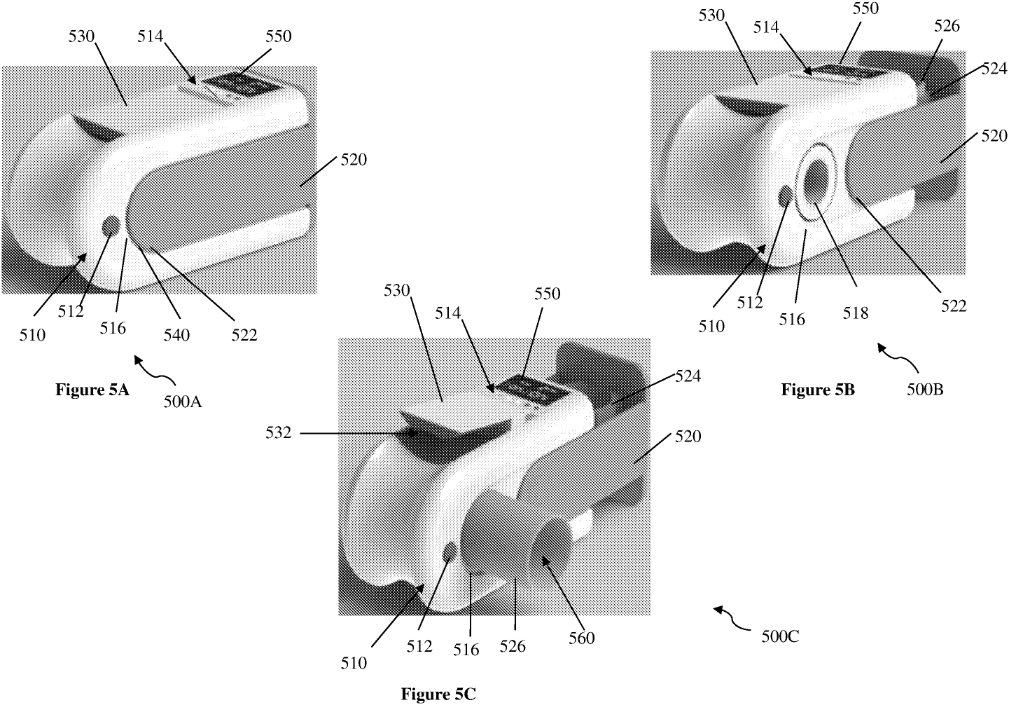

FIGS. 5A-5C depict patient monitoring device 500 in conformation A, B, and C (500A, 500B, and 500C, respectively). Conformation 500A of FIG. 5A depicts a closed conformation of patient monitoring device 500. Device body 510 includes power button 452 (for turning patient monitoring device 500 on and off). In conformation 500A, device body 510 can be seen slidingly coupled with housing 520, such that edge 522 of housing 520 and edge 516 of device body 510 meet at interface 540. Device body 510 further includes LED array 514, which is positioned next to display 550. It is contemplated that LED array 514 is used to communicate a status of patient monitoring device 500 (e.g., ready for use, sensor in use, data analysis pending, error in data, etc). In addition, display 550 is used to display results from spirometry or pulse oximetry tests. Hatch 530, positioned toward the top of device body 510, can be seen in a depressed, closed conformation, flush with device body 510. In conformations 500A, B, and C, Hatch 530 is held in the closed position by a tensioning mechanism (e.g., spring, etc). Hatch 530 opens upon insertion of the patient’s Index Finger. Hatch 530 closes upon removal of the patient’s Index Finger.

It is contemplated that conformation 500A is the conformation patient monitoring device 500 will be in when not used by a patient. It should be appreciated that in conformation 500A, the spirometer is not accessible in patient monitoring device 500. However, in conformation 500A, the pulse oximeter is operational upon insertion of the patient’s Index Finger into Hatch 530 and after activation of the power button 512.

Conformation 500B of FIG. 5B depicts an partially opened conformation of patient monitoring device 500. All similarly numbered components are as described for FIG. 5A. In conformation 500B, housing 520 has slid away from body 510, such that edges 522 and 516 no longer meet at an interface. In conformation 500B, air channel 518 of body 510 is now exposed, as are cradle 524 and tube 526. Hatch 530 remains in a depressed, closed conformation, flush with device body 510. Tube 526 can now be removed from cradle 524 and inserted into air channel 518 to provide an operational spirometer (e.g., airflow sensor). Thus it should be apparent that patient monitoring device 500 is partially operational in conformation 500B because a spirometer cannot be used. However, in conformation 500B, the pulse oximeter is operational upon insertion of the patient’s Index Finger into Hatch 530 and after activation of the power button 512.

Conformation 500C of FIG. 5C depicts an open, fully operational conformation of patient monitoring device 500. All similarly numbered components are as described for FIG. 5A. In conformation 500C, tube 526 is removed from cradle 524 and inserted into air channel 518 to provide an operational spirometer 560. Further, hatch 530 is now in an open conformation, no longer flush with device body 510, and instead reveals pulse oximeter 532. Thus, it should be appreciated that patient monitoring device 500 is fully operational in conformation 500C, because both spirometer 560 and pulse oximeter 532 are accessible and can be used to record respiratory parameter values and oxygenation parameter values.

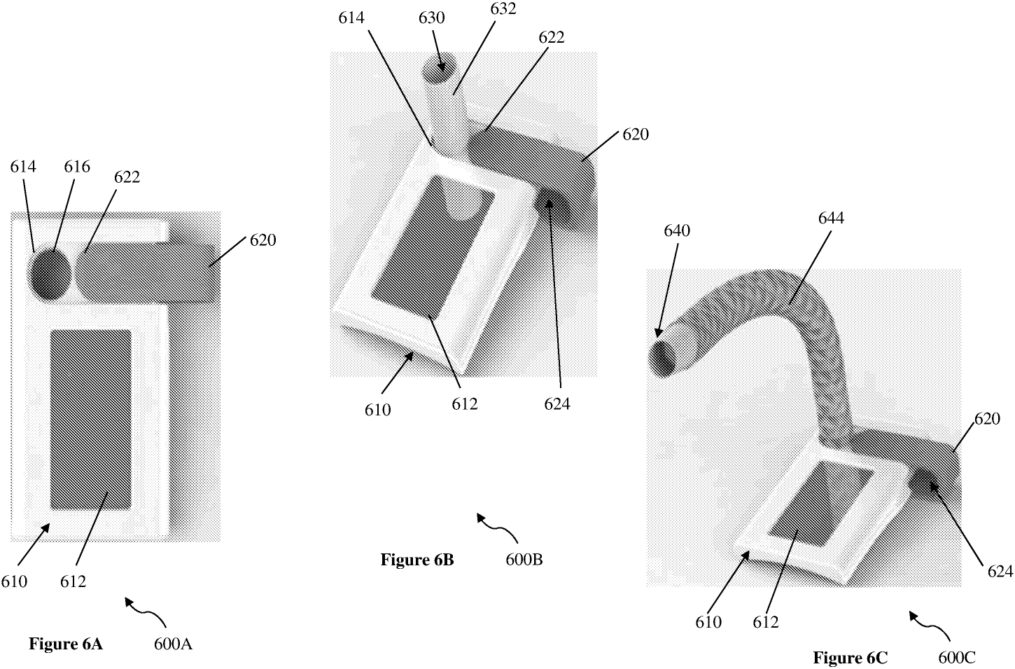

FIGS. 6A-6C depict patient monitoring device 600 in conformation A, B, and C (600A, 600B, and 600C, respectively). Conformation 600A of FIG. 6A depicts an open, partially operational conformation of patient monitoring device 600. Device body 610 includes display 612, which is used to communicate a status of patient monitoring device 600 (e.g., ready for use, sensor in use, data analysis pending, error in data, etc) to the user, as well as to display results from spirometry or pulse oximetry tests. In conformation 600A, device body 610 can be seen slidingly coupled with housing 620, such that edge 622 of housing 620 and edge 614 of body 610 do not meet at an interface. Instead, air channel 616 is exposed.

Conformation 600B of FIG. 6B depicts an open, operational conformation of patient monitoring device 600. All similarly numbered components are as described for FIG. 6A. In conformation 600B, tube 632 has been inserted into air channel 616, thus yielding spirometer 630 operational spirometry tests. Further, pulse oximeter 624 is accessible for oximetry tests. Thus it should be apparent that patient monitoring device 600 is fully operational in conformation 600B because spirometer 630 and pulse oximeter 624 can be used to record respiratory parameter values and oxygenation parameter values.

Conformation 600C of FIG. 6C depicts is an alternative operational conformation of patient monitoring device 400. All similarly numbered components are as described for FIG. 4A. In conformation 600C, collapsible hose 644 has been extracted from air channel 616 to provide an operational spirometer 640. Thus it should be apparent that patient monitoring device 600 is fully operational in alternative conformation 600C because spirometer 640 and pulse oximeter 624 can be used to record respiratory parameter values and oxygenation parameter values.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C .... and N, the text should be interpreted as requiring only one element from the group, not A plus N, or B plus N, etc.

What is claimed is:

1. A method of facilitating delivery of healthcare to a person, comprising:
using a monitoring device to derive a first set of respiratory data from the person;
using a processor to autonomously derive from the first set of respiratory data (1) a characterization of a health-related condition of the person, and (2) a therapy regimen related to the characterization;
using a user interface to instruct the person to perform a therapy regimen for the health-related condition; and
wherein each of the monitoring device, the processor, and the user interface cooperate as components of a mobile system.

2. The method of claim 1, wherein the monitoring device comprises a spirometer.

3. The method of claim 1, wherein the first set of respiratory data comprises a breathing pattern.

4. The method of claim 1, further comprising the processor autonomously deriving a second characterization of the health-related condition of the person, subsequent to the person performing the therapy regimen.

5. The method of claim 1, further comprising the processor autonomously deriving a second characterization of the health-related condition of the person, subsequent to the person performing an exercise test.

6. The method of claim 1, further comprising the processor triggering an instruction to the patient to retake a reading based upon a validity error.

7. The method of claim 1, further comprising using the user interface to instruct the person that the first set of respiratory data is within a healthy range.

8. The method of claim 1, wherein the health-related condition comprises a disease.

9. The method of claim 1, further comprising using the user interface to instruct the person to seek emergency health services.

10. The method of claim 1, further comprising using wirelessly conveying the first set of respiratory data to a healthcare professional.

11. The method of claim 1, further comprising wirelessly conveying the first set of respiratory data to a healthcare professional in accordance with provisions of the Health Insurance Portability and Accountability Act (HIPAA).

12. The method of claim 1, further comprising identifying the health-related condition as a false positive based on a demographic of the person.

13. The method of claim 1, further comprising identifying the health-related condition as a false positive based on an environmental characteristic of the person.

14. The method of claim 1, wherein the processor additionally uses data from a pulse oximeter to derive at least one of the characterization and the therapy regimen.

15. The method of claim 1, wherein the processor additionally uses data from a respiratory sound of the person to derive at least one of the characterization and the therapy regimen.

16. The method of claim 1, wherein the processor additionally uses data from a respiratory sound of the person to verify the identity of the person.
